(19) United States
(12) Patent Application Publication
(10) Pub. No.: US 2004/0176404 A1
(43) Pub. Date: Sep. 9, 2004

(54) Methods and reagents for treating or preventing atherosclerosis and diseases associated therewith

(76) Inventor: Chalom B. Sayada, Luxembourg City (LU)

Correspondence Address: CLARK & ELBING LLP, 101 FEDERAL STREET, BOSTON, MA, 02110, US

(21) Appl. No.: 10/735,344

(22) Filed: Dec. 11, 2003

Related U.S. Application Data
(60) Provisional application No. 60/433,379, filed on Dec. 12, 2002.

(51) Int. Cl. A61K 31/4745

(52) U.S. Cl. 514/291

(57) ABSTRACT

The invention features a method for treating, preventing, or reducing the development of an atherosclerosis-associated disease in a patient by administering to the patient a rifamycin in an amount effective to treat, prevent, or prevent the development of the atherosclerosis-associated disease in the patient.

### CROSS REFERENCE TO RELATED APPLICATIONS

This application claims the benefit of the filing date of U.S. provisional application, U.S.S.N. 60/433,379, filed Dec. 12, 2002.

### BACKGROUND OF THE INVENTION

The invention relates to the field of bacterial infections.

Atheroclerosis-associated diseases are the largest single cause of premature death in the western world. Although predisposition to atherosclerosis has traditionally been associated with age, social, and economic factors, a growing body of evidence has recently implicated various bacteria as causative agents. One such bacterium is Chlamydia (C.) pneumoniae, a pathogen involved in acute and chronic respiratory infections. On the basis of its presence in atherosclerotic lesions and its absence in healthy artery tissues, C. pneumoniae has been implicated in the initiation and pathogenesis of atherosclerosis. It has been suggested that C. pneumoniae lodges in the walls of blood vessels remaining there for years. The chronic inflammation triggered by the persistent bacterial infection within the arterial walls may induce host macrophages to remove fat, cholesterol, and other deposits from the vessel walls, ultimately causing arterial irritation and scarring. The consequent build-up in arterial plaques can foster blood clots and impede circulation, thus increasing susceptibility to a number of disorders, including heart attacks and strokes.

While the administration of antibiotics has been suggested to treat or prevent atherosclerosis-associated diseases by eradicating C. pneumoniae infection in arteries, little success has been reported. Thus, there is a need for improved methods for treating or preventing the development of atherosclerosis in patients infected with C. pneumoniae.

### SUMMARY OF THE INVENTION

In general, the present invention is based on our discovery that rifamycins are uniquely capable of reaching and eradicating C. pneumoniae present in foam cells or macrophages found in the arterial fatty streaks that are associated with atherosclerosis.

Accordingly, the invention features a method of treating, reducing, or preventing the development of an atherosclerosis-associated disease in a patient by administering to the patient a rifamycin in an amount effective to treat, reduce, or prevent the development of the atherosclerosis-associated disease in the patient. Prior to the administration of the rifamycin, the patient may be diagnosed as having the atherosclerosis-associated disease (or being at increased risk of developing such disease) or as having macrophages or foam cells infected with C. pneumoniae.

The invention also features a method of reducing the level of C-reactive protein in a patient in need thereof by administering to the patient a rifamycin in an amount effective to reduce the level of C-reactive protein in the patient. The patient may not have been diagnosed as having a bacterial infection (e.g., an infection that can be treated by administration of a rifamycin). Furthermore, the patient may have been diagnosed as having macrophages or foam cells infected with C. pneumoniae.

The invention also features a method of reducing C. pneumoniae replication in macrophages or foam cells in a patient in need thereof by administering to the patient a rifamycin in an amount effective to reduce C. pneumoniae replication in macrophages or foam cells in the patient.

The invention also features a method of treating a persistent C. pneumoniae infection in macrophages or foam cells in a patient by administering to the patient a rifamycin in an amount effective to treat the Chlamydia pneumoniae infection in macrophages or foam cells in the patient.

In any of the foregoing aspects, the dosage of rifamycin normally ranges between 0.001 mg to 100 mg, preferably between 1 mg-50 mg, or more preferably between 2-25 mg. The rifamycin may be given daily (e.g., a single oral dose of 0.001 mg to 100 mg/day, preferably 2.5 to 25 mg/day) or less frequently (e.g., a single oral dose of 5 mg/week, 12.5 mg/week, or 25 mg/week). Treatment may be given for a period of one day to one year, or longer. In one embodiment, a rifamycin is administered at an initial dose of 2.5 mg to 100 mg for one to seven consecutive days, followed by a maintenance dose of 0.005 mg to 10 mg once every one to seven days for one month, one year, or even for the life of the patient.

The dosage of rifampin, rifabutin, rifapentin, or rifaximin normally ranges between 50 to 1000 mg/day. These rifamycins may be given daily (e.g., a single oral dose of 50 to 600 mg/day) or less frequently (e.g., a single oral dose of 50, 100, or 300 mg/week). Treatment may be administered for a period of one day to one year, or even longer. In one embodiment, a rifamycin is administered at an initial dose of 600 mg to 2000 mg for one to seven consecutive days, followed by a maintenance dose of 100 mg to 600 mg once every one to seven days for one month, one year, or even for the life of the patient.

If desired, a rifamycin may be administered in conjunction with one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., azithromycin, clarithromycin, erythromycin, roxythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These secondary therapeutic agents may be administered within 14 days, 7 days, 1 day, 12 hours, or 1 hour of administration of a rifamycin, or simultaneously therewith. The additional therapeutic agents may be present in the same or different pharmaceutical compositions as the rifamycin of the invention. When present in different pharmaceutical compositions, different routes of administration may be used. For example, rifalazil may be administered orally, while a second agent may be administered by intravenous, intramuscular, or subcutaneous injection.

By “atherosclerosis” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

By “atherosclerosis-associated disease” is meant any disorder that is caused by or is associated with atherosclerosis. Typically, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis).

A patient who is being treated for an atherosclerosis-associated disease is one who a medical practitioner has diagnosed as having such a disease. Diagnosis may be done by any suitable means. Methods for diagnosing atherosclerosis by measuring systemic inflammatory markers are described, for example, in U.S. Pat. No. 6,040,147, hereby incorporated by reference. Diagnosis and monitoring may employ an electrocardiogram, chest X-ray, echocardiogram, cardiac catheterization, ultrasound (for the measurement of vessel wall thickness), or measurement of blood levels of CPK, CPK-MB, myoglobin, troponin, homocysteine, or C-reactive protein. A patient in whom the development of an atherosclerosis-associated disease is being prevented is one who has not received such a diagnosis. One in the art will understand that these patients may have been subjected to the same tests (electrocardiogram, chest X-ray, etc.) or may have been identified, without examination, as one at high risk due to the presence of one or more risk factors (e.g., family history, hypertension, diabetes mellitus, high cholesterol levels). Thus, prophylactic administration of a rifamycin is considered to be preventing the development of an atherosclerosis-associated disease.

An atherosclerosis-associated disease has been treated or prevented when one or more tests of the disease (e.g., any of the those described above) indicate that the patient's condition has improved or the patient's risk reduced. In one example, a reduction in C-reactive protein to normal levels indicates that an atherosclerosis-associated disease has been treated or prevented.

An alternative means by which treatment or prevention is assessed includes determination of the presence of an infection of C. pneumoniae. Any suitable method may be employed (e.g., determination of C. pneumoniae in blood monocytes or in the atheroma itself (e.g., in macrophages or foam cells present in the fatty streak), or detection of C. pneumoniae DNA, C. pneumoniae RNA, or antibodies to C. pneumoniae in a biological sample from the patient).

The invention also features a stent coated with a rifamycin. The stent can be, e.g., a wire mesh tube used to hold open an artery. Stents are typically inserted following angioplasty.

Rifamycins are compounds characterized by a chromophoric naphthohydroquinone group spanned by an aliphatic bridge. Exemplary rifamycins are rifalazil (3′-hydroxy-5′-(4-isobutyl-1-piperazinyl) benzoxazinorifamycin; also known as KRM-1648), rifampin, rifabutin, rifapentin, and rifaximin. Other rifamycins are disclosed in U.S. Pat. Nos. 4,690,919; 4,983,602; 5,786,349; 5,981,522; 6,316,433 and 4,859,661, and U.S. Patent Application Nos. 60/341,130 and 60/341,591, each of which is hereby incorporated by reference.

### DETAILED DESCRIPTION OF THE INVENTION

We have discovered that administration of a rifamycin is effective to treat, reduce, or prevent the development of an atherosclerosis-associated disease in a patient.

According to the present invention, a rifamycin may be administered by any route that results in an effective amount reaching the atheroma or the foam cells (lipid-laden macrophages that constitute the fatty streak). The rifamycin is normally administered in an amount ranging between 0.001 to 100 mg/day. The rifamycin may be given daily (e.g., a single oral dose of 2.5 to 25 mg/day) or less frequently (e.g., a single oral dose of 5, 12.5, or 25 mg/week). Patients may be treated for a period of one day to one year, or even longer. It may be desirable to commence therapy with a higher initial dose, followed by a lower maintenance dose. The rifamycin may be contained in any appropriate amount in any suitable carrier substance and is generally present in an amount of 1-95% by weight of the total weight of the composition. The pharmaceutical composition can generally be formulated according to conventional pharmaceutical practice (see, e.g., Remington: The Science and Practice of Pharmacy (20th ed.), ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York).

Rifamycins include rifalazil, rifampin, rifabutin, rifapentin, rifaximin, and compounds described by formula I:

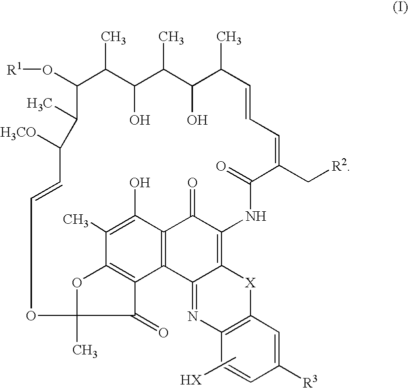

In formula I, X represents an oxygen atom or a sulfur atom, R¹ represents a hydrogen or an acetyl group, R² represents a hydrogen or hydroxyl group, and R³ represents a group expressed by the formula:

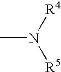

such that each of R⁴ and R⁵ is, independently, an alkyl group having 1 to 7 carbon atoms, or alternatively, R⁴ and R⁵ may combine to form a 3-8 membered cyclic system.

R³ may also be represented by a group expressed by the formula:

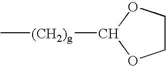

in which g represents an integer between 1 and 3. R³ may alternatively represent a group expressed by the formula:

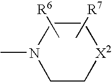

such that each of R⁶ and R⁷ is, independently, a hydrogen atom or an alkyl group having 1 to 3 carbon atoms, X² represents an oxygen atom, a sulfur atom, or a carbonyl group. X² may also be a group expressed by the formula:

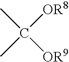

in which each of R⁸ and R⁹ is, independently, a hydrogen atom, or an alkyl group having 1 to 3 carbon atoms, or R⁸ and R⁹ may be, in combination with each other, represented by —(CH₂)k— in which k represents an integer between 1 and 4. X² may also be represented by a group expressed by the formula:

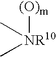

in which m represents 0 or 1, R¹⁰ represents a hydrogen atom, an alkyl group having 1 to 6 carbon atoms, or —(CH₂)ₙX³ in which n represents an integer between 1 and 4, and X³ represents an alkoxy group having 1 to 3 carbon atoms, a vinyl group, an ethynyl group. Alternatively, X² represents a group expressed by the formula:

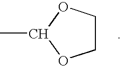

In addition to being administered one or more rifamycins, a patient being treated according to the present invention may further be administered second therapeutic agents such as anti-inflammatory agents, antibacterial agents, platelet aggregation inhibitors, anticoagulants, or lipid lowering agents.

### OTHER EMBODIMENTS

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in microbiology or related fields are intended to be within the scope of the invention.

What is claimed is:

1. A method of treating, preventing, or reducing the development of an atherosclerosis-associated disease in a patient in need thereof, said method comprising administering to said patient a rifamycin in an amount effective to treat, prevent, or reduce the development of said atherosclerosis-associated disease in said patient.

2. The method of claim 1, wherein said rifamycin is administered in an amount ranging between 0.001 and 100 mg.

3. The method of claim 2, wherein said rifamycin is administered in an amount ranging between 1 and 50 mg.

4. The method of claim 1, wherein said rifamycin is administered in an amount ranging between 5 and 25 mg/week.

5. The method of claim 4, wherein said rifamycin is administered in an amount ranging between 2.5 and 25 mg/day.

6. The method of claim 1, wherein said rifamycin is administered at an initial dose of 2.5 mg to 100 mg for one to seven consecutive days, followed by a maintenance dose of 0.005 to 10 mg once every one to seven days.

7. The method of claim 1, wherein said patient is further administered a second therapeutic agent.

8. The method of claim 7, wherein second therapeutic agent is an anti-inflammatory agent, antibacterial agent, platelet aggregation inhibitor, anticoagulant, antipyretic, or lipid-lowering agent.

9. The method of claim 8, wherein said anti-inflammatory agent is ibuprofen, meloxicam, celecoxib, rofecoxib, aspirin, dexamethasone, methylprednisolone, prednisolone, or prednisone.

10. The method of claim 8, wherein said antipyretic is acetaminophen.

11. The method of claim 8, wherein said antibacterial agent is azithromycin, clarithromycin, erythromycin, gatifloxacin, levofloxacin, amoxicillin, or metronidazole.

12. The method of claim 8, wherein said lipid-lowering agent is a statin.

13. The method of claim 12, wherein said statin is atorvastatin, rosuvastatin, lovastatin, simvastatin, pravastatin, cerivastatin, or fluvastatin.

14. The method of claim 1, wherein said atherosclerosis-associated disease is coronary artery disease, myocardial infarction, angina pectoris, stroke, cerebral ischemia, intermittent claudication, gangrene, mesenteric ischemia, temporal arteritis, or renal artery stenosis.

15. The method of claim 1, wherein, prior to administration of said rifamycin, said patient is diagnosed as having said atherosclerosis-associated disease.

16. The method of claim 1, wherein said patient has not been diagnosed as having a bacterial infection.

17. A method of reducing the level of C-reactive protein in a patient identified as having increased levels of C-reactive protein, said method comprising administering to said patient a rifamycin in an amount sufficient to reduce the level of C-reactive protein.

18. The method of claim 17, wherein said method further comprises the step of periodically monitoring the level of C-reactive protein in said patient following administration of said rifamycin.

19. The method of claim 17, wherein said patient has not been diagnosed as having a bacterial infection.

20. A method for reducing Chlamydia pneumoniae replication in macrophages or foam cells in a patient in need thereof, said method comprising administering a rifamycin to said patient in an amount effective to reduce Chlamydia pneumoniae replication in macrophages or foam cells in said patient.

21. A method for treating a persistent Chlamydia pneumoniae infection in macrophages or foam cells in a patient, said method comprising administering a rifamycin to said patient in an amount effective to treat said Chlamydia pneumoniae infection in macrophages or foam cells in said patient.
